(19) United States
(12) Patent Application Publication
(10) Pub. No.: US 2010/0114124 A1
(43) Pub. Date: May 6, 2010

(54) Method and apparatus for partioning an organ within the body

(76) Inventors: Brian Kelleher, San Diego, CA (US); Paul Swain, London (GB)

Correspondence Address: SCOTT EVANS, 1252 COUNTRY HILLS DR., SANTA ANA, CA, 92705, US

(21) Appl. No.: 11/497,099

(22) Filed: Aug. 1, 2006

Related U.S. Application Data
(60) Provisional application No. 60/705,087, filed on Aug. 3, 2005.

(51) Int. Cl. A61B 17/00

(52) U.S. Cl. 606/151

(57) ABSTRACT

The preferred methods and devices described herein relate to devices and methods for joining segments of soft tissue together. More particularly this invention relates to partitioning a body cavity or organ by joining together portions of the organ interior walls. This securement is particularly useful in gastric volume reduction surgery whereby the volume of the stomach is reduced by partitioning the stomach into a smaller pouch.

### CLAIM OF PRIORITY

This application claims priority under 35 U.S.C. §119(e) to U.S. Provisional Application No. 60/705,087, filed Aug. 3, 2005, the entire contents of which are hereby expressly incorporated by reference.

### BACKGROUND

1. Field of the Invention

The present invention relates to devices and methods for joining segments of soft tissue together. More particularly this invention relates to partitioning a body cavity or organ by joining together portions of the organ interior walls.

2. Description of the Related Art

Often segments of soft tissue are brought together for the purpose of reseeting tissue, providing anchors for other devices and for creating walls or partitions within a body cavity or an organ having a lumen. Sometimes a single wall of tissue is folded and brought together and other times two portions of soft tissue are grasped separately and then the two portions are brought into close proximity to each other and then joined together either permanently or temporarily. The joining of portions of soft tissue has traditionally been done using clamping, banding, suturing or stapling devices. However, joining segments of tissue together whereby some of these may be exposed to tension post-operatively often does not hold up over time. For example, when two discrete segments of the stomach are sewn together the sutures that hold the segments together are in tension post-operatively. In order to prevent the sutures or other fastening devices from pulling through the stomach wall over time, the sites where the devices puncture the outer wall of the stomach are sometimes reinforced with sections of tear-resistant material called pledgets; otherwise, other techniques must be employed to prevent pull out.

The placement of staples, sutures and the use of pledgets is not always possible especially when securing the wall of an organ that has a surface not easily accessible during the procedure. As an example, when performing an endoluminal gastroplasty procedure, that is, when sewing the wall of the stomach to itself from within the lumen of the stomach to reduce its volume, only the inner wall is accessible. Sutures that are placed through the wall can be strain-relieved with a pledget or similar device only along the inner surface of the wall, but not along the outer wall (unless a pledget or similar device is passed through the wall, which is generally not practical). Furthermore, when fastening devices such as sutures are exposed to tension, as is the case when a gastroplasty procedure is done to create a gastric restriction, the fastening devices generally pull out over time. Additionally, many procedures requiring endolumenal tissue apposition and securement inside the interior space of an organ like the stomach suffer from the need for impractically complex tissue manipulation mechanisms.

The only method that has proven useful to create a wall-to-wall adhesion (i.e., from the anterior wall of the stomach to the posterior wall of the stomach) is the multiple-row stapler. However these staples are applied from outside the stomach and thus require some form of surgical invasion of the peritoneal space outside the stomach. The mechanism of action of these staplers is related to the wide band of injury that occurs along the staple line, resulting from a combination of an initial crushing injury followed by a band of necrosis resulting from isehemia induced by the wide row of staples. The piercing effect of the staples may also be important, as may be the foreign body response created by the staples.

To simulate this type of wall-to-wall securement from within the interior space of an organ like the stomach, one alternative is to secure an invaginated fold from the posterior wall to an invaginated fold from the anterior wall. This approach has been disclosed in the pending patent application 2005/0055038 filed Sep. 9, 2003 entitled “Device and Method for Endoluminal Therapy” the entire contents of which are included by reference. This present application relates to an improvement on the methods and devices disclosed in the 2005/0055038 application that are believed to make the procedure more practical and more durable.

There is therefore a need for devices and methods that enable wall to wall securement with reduced chance of detachment occurring post-operatively. More specifically, there is a need for devices and methods that join tissue walls together, provide pressure on the joint and promote inner tissue layer intermingling. Additionally, these tissue securement devices need to be delivered endoscopically, as through a rigid endoscope, or endoluminally, as through a flexible endoscope.

### BRIEF SUMMARY OF THE INVENTION

The preferred methods and devices described herein provide for securing a fold or folds of a wall of an organ together. This securement is particularly useful in gastroplasty surgery whereby partitions are created at various locations in the stomach for the purpose of treating conditions such as obesity and gastro-esophageal reflux disease (GERD). In one form of such a procedure, a line of wall-to-wall securements are made along a line from the cardia of the stomach to the lesser curvature. This line of new tissue unions can form a stomach pouch with a restrictive outlet, mimicking similar conventional surgical procedures performed to treat obesity.

One aspect of the invention is a device to secure folds of an interior wall of an organ together. The device comprises at least one grasping element suitable for grasping one portion of the wall to form a first fold, and at least a second grasping element that is suitable for grasping a second portion of the wall to form a second fold and also suitable for positioning the second fold alongside the first fold with the folds in a side by side relationship. Another aspect of the invention includes a clamping member to secure the folds together.

Another embodiment of the invention is a device to partition a portion of the interior space of an organ having at least one grasping element suitable for grasping one portion of a wall of the organ to form a first fold and having at least a second grasping element suitable for grasping a second portion of a wall of the organ to form a second fold. The two folds are positioned alongside each other with the folds in a side by side relationship and an elongate clamping member is used to secure the folds together.

Another aspect of the invention is a method to secure the folds of an interior wall of an organ together including grasping a first portion of the wall to form a first fold and grasping a second portion of the wall to form a second fold with the folds in a side by side relationship. The method further comprises securing the folds together using at least one elongate clamping member positioned about the side by side folds.

In another aspect of the invention is a method to create a passageway along an interior wall of the stomach which comprises a) grasping one portion of the wall to form a first fold and b) grasping a second portion of the wall to form a second fold and positioning the second fold alongside the first fold with the folds in a side by side relationship and c) securing the folds together. The method further comprises repeating steps a, b and c to form a series of secured folds of the wall along a line running from the cardia towards the lesser curvature of the stomach. The method further comprises clamping the folds with an elongate clamping member wherein one end of the elongate clamping member is positioned against the cardia as a pivot point such that when the clamping member is positioned over the folds, the pivot point maintains at least one end of the clamping device against the stomach wall. The method further comprises a detachable pusher element attached to the clamping member that is adapted to retain an opposite end of the elongate clamping member in a desired position while positioning the clamping member over the folds.

Certain objects and advantages of the invention are described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the present invention herein disclosed. However, despite the foregoing discussion of certain embodiments, only the appended claims (and not the present summary) are intended to define the invention. The summarized embodiment, and other embodiments of the present invention, will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

FIG. 1 is a perspective view showing the desired location of an approximated fold of stomach tissue to create a partition in the stomach;

FIG. 2 is a perspective view of a clamping member;

FIG. 3 is a perspective view of another embodiment of a clamping member;

FIG. 4A is a drawing of a clamping member showing an articulated hinge;

FIG. 4B is a drawing of a clamping member that is bent at the hinge point;

FIG. 4C is a drawing of a clamping member placed into the mouth of a patient;

FIG. 5 is a drawing of a grasping element showing a grasping head, connecting element, and actuator assembly;

FIG. 6 is a perspective view showing a clamping member positioned about two grasping elements;

FIG. 7A is a drawing of a pushing element detachably coupled to a clamping member;

FIG. 7B is a view of grasping elements and a clamping member shown in the esophagus of a patient;

FIG. 8 is a cutaway view of a preferred embodiment of grasping elements and the clamping member in a stomach;

FIG. 9 is a drawing showing several folds of soft tissue after being drawn up into the clamping member;

FIG. 10 is a drawing showing the proper placement of the graspers to effect the desired partition in a stomach;

FIG. 11A is a section view of a retention member on the clamping member;

FIG. 11B is a section view of an alternative embodiment of the retention member, showing the tissue flaps being pulled through it;

FIG. 11C is a section view of the retention member shown in FIG. 11b, showing the engagement of the retention member with the tissue flaps after they have been released;

FIG. 12 is a drawing of a user activated clamping mechanism;

FIG. 13 is a drawing of a clamping member with an integral stapling mechanism;

FIG. 14 is a side view of a clamping member with an interrupted clamping surface;

FIG. 15 is a drawing of a clamping member with integral tissue cutter;

FIG. 16A is a drawing showing designs to enhance the adhesion of the clamped layers;

FIG. 16B is a drawing showing alternate designs to enhance the adhesion of the clamped layers;

FIG. 16C is a drawing showing alternate designs to enhance the adhesion of the clamped layers;

FIG. 17A is a drawing of an alternate clamping member used to approximate tissue near the fundus;

FIG. 17B is a side view of a clamping member securing three folds of tissue;

FIG. 18 is a drawing of a clamping element and spacer to maintain an opening in the stomach;

FIG. 19A is a drawing of an integral clamp and grasping element with the grasping element retracted;

FIG. 19B is a drawing of an integral clamp and grasping element with the grasping element deployed;

FIG. 20 is an end view of an endoscope showing a typical auxiliary channel for the positioning of accessory elements as described in prior art;

FIG. 21A is an end view of an endoscope with a preferred embodiment auxiliary channel for the positioning of multiple grasping elements;

FIG. 21B is a side view of an endoscope showing two instruments sharing a single working channel;

FIG. 22 is a drawing showing a method to locate the lesser curvature in the stomach;

FIG. 23A is a drawing of an articulated clamping member in a stomach;

FIG. 23B is a drawing of the tissue union line as a result of using an articulated clamping member;

FIG. 24A is a side view of an apparatus having a pulley element;

FIG. 24B is a close-up view of an apparatus having a pulley element;

FIG. 25A is a schematic view of a clamp assembly;

FIG. 25B is a schematic view of the clamp assembly of FIG. 25A in a stomach;

FIG. 25C is a schematic view of the clamp assembly positioned over a partition in the stomach.

### DETAILED DESCRIPTION OF THE INVENTION

The devices and methods described may provide a better way to partition the interior of an organ or body cavity. The resultant remodeled interior space will be formed by one or a series of wall-to-wall tissue unions that may be formed using graspers and securement devices. As shown in FIG. 1, one preferred goal is to partition the stomach 10 having an interior space 12 such that a separate region 14 is formed. At the entrance to the stomach 10 is located the distal end 16 of the esophagus. The distal end 16 of the esophagus terminates at the lower esophageal sphincter 18 before entering the stomach 10. The upper portions of the stomach are defined by the fundus 20, cardia 22 and the lesser curvature of the stomach 24, which is the shortest wall between the lower esophageal sphincter 18 and the distal opening of the stomach, the pylorus 26. The approximate position of a partition wall 28 is shown. The present invention is designed to be able to form partition wall 28 by drawing together folds of tissue from the interior of the stomach and then clamping them together. The basic steps consist of a) grabbing portions of the anterior and posterior stomach or other organ wall with tissue grasping elements, b) positioning a clamping device in the desired location along the stomach wall c) pulling the grasping elements such that portions of the wall of the stomach or organ are pulled through the clamping device and d) then releasing the grasping elements.

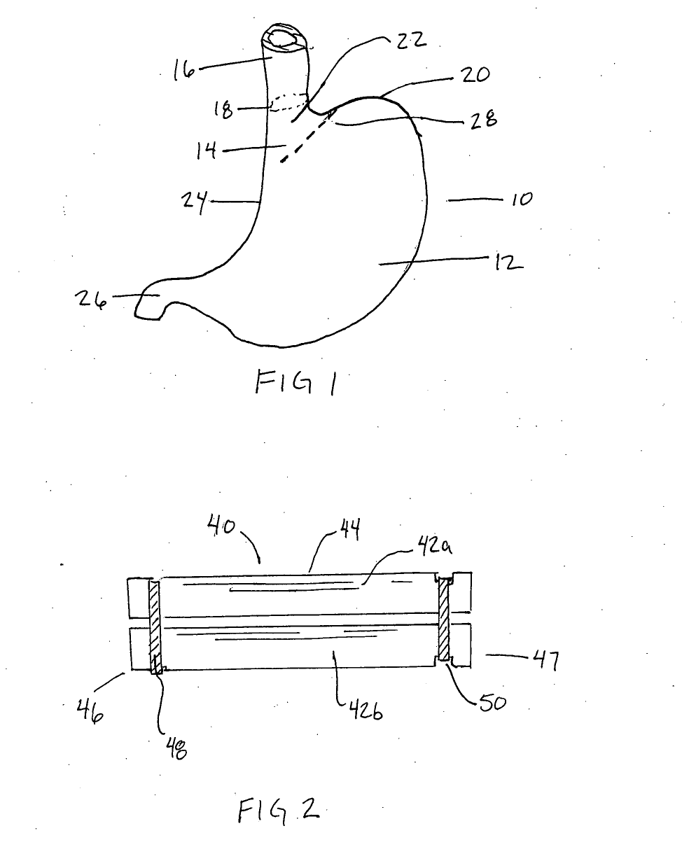

In a particular embodiment for creating a partition in the stomach, a clamping device 40 is shown in FIG. 2. In this embodiment the clamping device 40 is made from two elongated beam elements 42a and 42b. The beam elements 42a and 42b should preferably be constructed of strong material so that when tissue is clamped between the two beam elements 42a and 42b, the clamping force is evenly distributed across the length of the beam element. The beam elements may have rounded or chamfered ends 46 and 47 so as to ease the insertion of the clamping device through a hollow passageway such as the esophagus. In one version the beam may be round and rotate like a roller but this rolling is not required for proper function. The clamping device 40 is secured at both ends 46 and 47 with a retainer 48. The retainer 48 may be captured in a notch 50 formed near the ends 46 and 47 of the beam elements 42a and 42b, The retainer may be made from elastomeric elements such as rubber bands or o-rings. Instead of an elastomeric element, other elements are readily conceivable to those knowledgeable in the art, such as metal springs, superelastic devices such as Nitinol and the like. The retainer 48 is designed to keep the beam elements 42a and 42b together in a closed configuration but ideally should allow the beam elements to separate to an open configuration whereby a small slot is opened between the beam elements. In this open configuration, the clamping device 40 can receive graspers attached to folds of tissue and then move to the closed configuration to securely clamp the folds of tissue together.

Referring now to FIG. 3, one end of clamping device 40 could be hinged 61 and the other ends 62 and 64 could be closeable with a closure mechanism such as a pin and clasp. As shown in FIG. 3, a pin 66 is located at one of the open ends 62 and a receiving receptacle or clasp 68 is positioned opposite on end 64. This embodiment shows a ratcheting option, which may not be required in all cases. Other closure mechanisms such as bayonet, slip fit, etc. are also possible. In this configuration, the clamping device 40 could be placed around a fold of tissue and then the two ends 62 and 64 closed by the operator by pushing on an actuator (not shown). The hinge 61 may be constructed as a conventional hinge with a hffigepin or other elements know to those skilled in the art, or it may be a living hinge, molded in one piece along with both beams.

Some important parameters are clamping force and the size and shape of the clamping surface. Clamping force needs to be enough to occlude the blood supply in the tissue, and this may range from less than one PSI to 20 PSI, by way of example. The clamping surface should have a width similar to that used for staplers, ranging from about 2 mm to 10 mm, and it may be substantially flat across the clamped tissue. Alternatively, it may have a stepped surface.

The clamping device 40 may be passed trans-esophageally to the stomach. It may also need to be long enough to create a full-width partition across the stomach, so preferably it should be from about 2 inches to about 4 inches in length. However it is difficult to pass something this long around the crycopharyngeal junction which is a location in the esophagus where the esophagus makes a severe turn accompanied with a slight narrowing. So it will preferably articulate as it passes through certain anatomical features. One approach is to have the clamping device 40 be hinged, so that it may articulate or bend in one place, and preferably in one direction only, but in no other places. It is important that the clamp is stiff along the plane that is parallel with the midline between the clamping elements (where the tissue will be clamped), so that the tissue is clamped with relatively even pressure across the clamping surfaces.

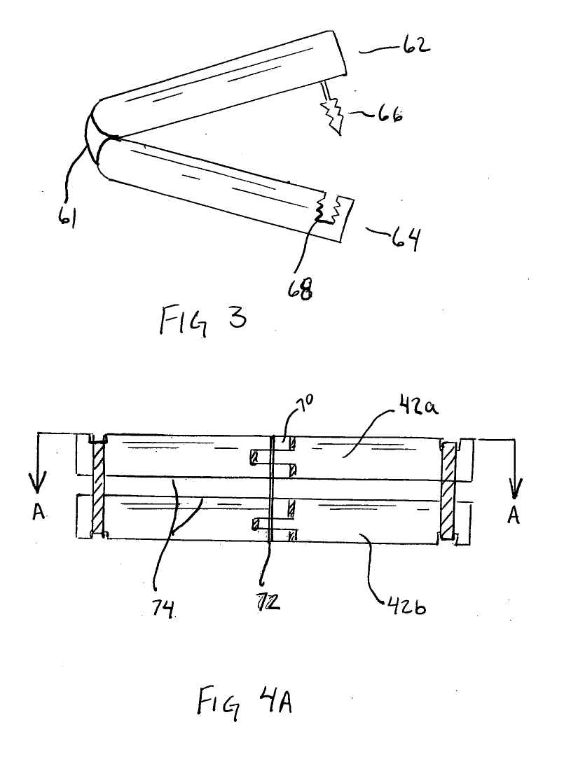

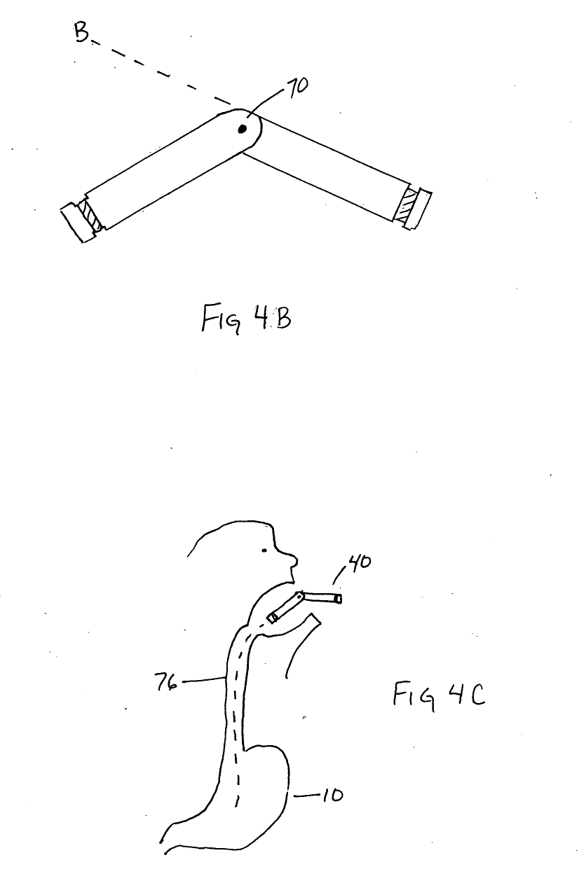

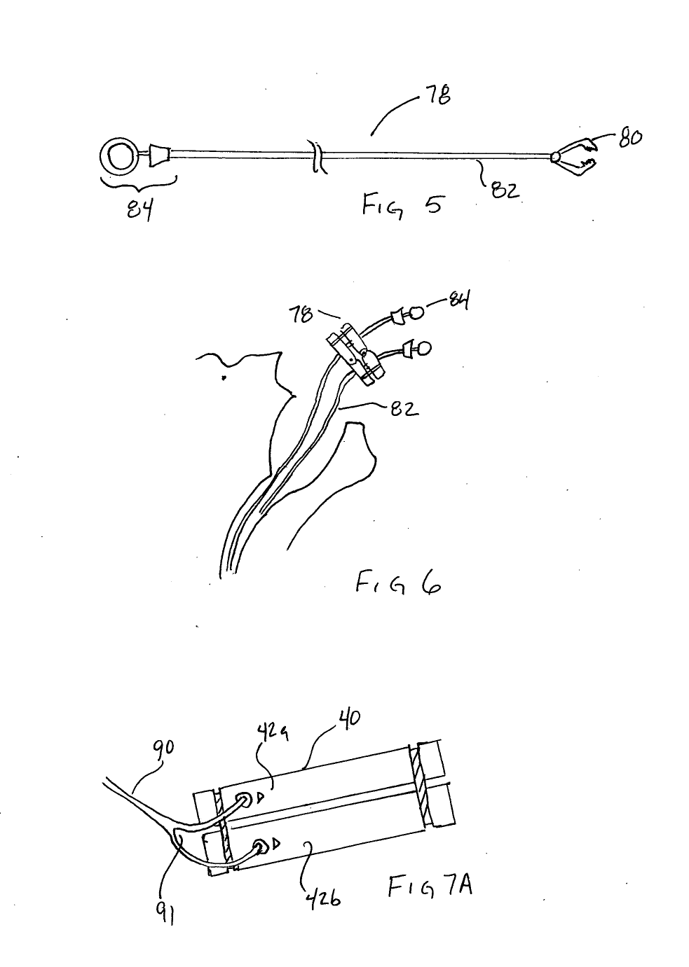

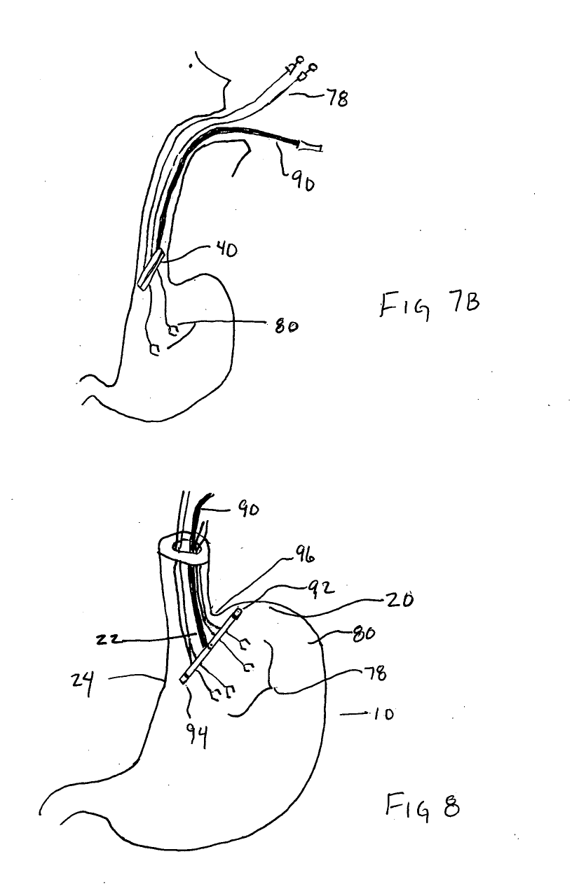

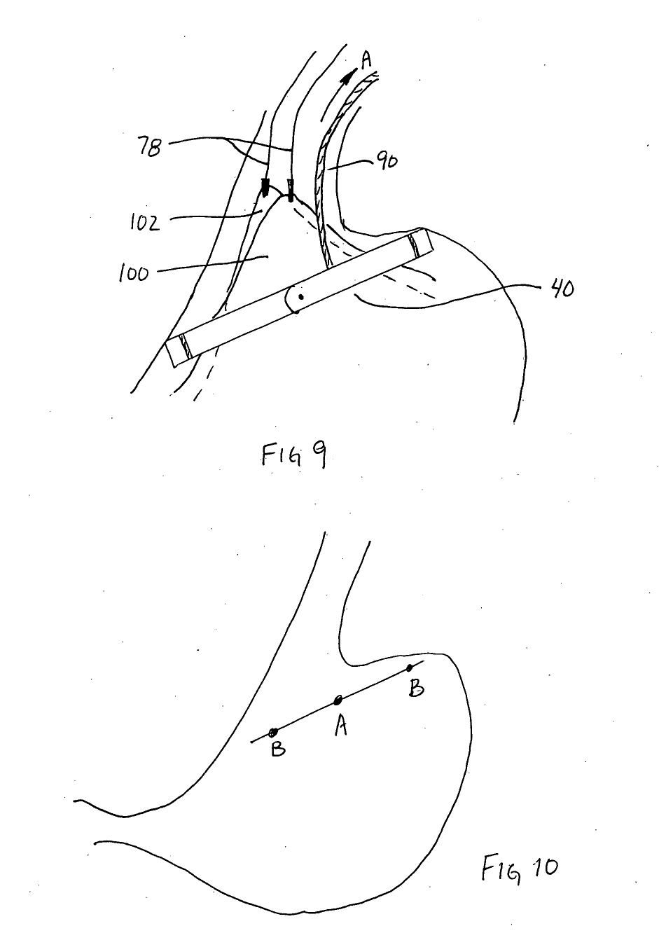

An example of a simple hinge design is shown in FIG. 4A. As shown the beam elements 42a and 42b are shown with a secondary hinge 70 and hinge pin 72. In this embodiment, the clamping device 40 remains rigid along clamping surfaces 74, but can bend in an orthogonal plane. This is better shown in FIG. 4B where the hinge is shown in a side view taken along plane A-A. Preferably the clamping device 40 cannot bend backwards from this position past the straight line B. In this regard the hinged clamping member resembles a locking knee. The reason for this feature is that it relates to the need to pull against the clamping device 40 without it buckling, as will be explained later. With this design, clamping device 40 can be delivered through the esophagus 76 to the stomach 10 and be able to navigate tortuous portions of the esophageal passageway.

In a preferred method, the grasping elements 78 are placed in desired locations under visual guidance by use of an endoscope or other means. In this method, the stomach 10 may be insufflated and the endoscope's articulation features may be used to position the grasping elements 78 in their desired locations. This method allows visual verification that the grasping elements 78 have adequately grasped the tissue, by pulling back on the graspers and testing the degree of grasping. The grasping elements 78 are comprised of a grasping head 80, a connecting element 82 and an actuator assembly 84 as shown in FIG. 5. Connecting element 82 is preferably flexible and torqueable. The grasping head 80 is shown as a clamping jaw that is activated with a linkage extending down the connecting element to the activator assembly which is a mechanical handle. However many other clamping head types are possible such as a corkscrew that is rotated and driven into the tissue with a spiral motion. The clamping head 80 may also be a barb or hook that snares the tissue and pulls the tissue against the hooked end. Other tissue grabbers common to those in the art are anticipated.

By way of example, either two or four grasping elements 78 may be used to create a stomach partition. When just two are used, one is used to grasp the middle of the desired partition zone on the anterior wall, and the other is used to grasp the middle of the desired partition on the posterior wall. When four are used, two grasping elements 78 are used to pull on the outer ends of the desired partition zone on the anterior wall, and two on the posterior wall, Preferably the grasping elements 78 have a locking feature, similar to a hemostat, that keeps a firm grasp of the tissue once the grasping elements 78 are properly positioned, without requiring constant actuating pressure from the operator.

In one embodiment of the invention, once the grasping elements 78 are in place, the clamping device 40 is slid into position in the stomach 10. Preferably the clamping device 40 is positioned around the connecting elements 82 of the graspers. When two grasping elements 78 are utilized, the grasping elements 78 are first applied to the tissue as has been described and then the clamping device 40 is placed around the grasping elements outside the patient's mouth as shown in FIG. 6. The clamping device 40 may be placed in a first open configuration to facilitate placement around the grasping element 78 or if it is normally open as with an open ended clamp similar to the one shown in FIG. 3 simply placed around the connecting elements 82 and then the clasp may be at least partially closed. The clamping device 40 is then slid along the connecting element, down the esophagus and into place in the stomach. It may be pushed along by a pusher element 90 as shown in FIG. 7A. In this embodiment the pusher is detachably connected to the elongated beams 42a and 42b at point D. The pusher element 90 may have a wishbone distal end portion 91 so that a single pusher element 90 may be attached to both beams. The wishbone should be large enough to accommodate any flap of tissue that may be drawn into the clamping device 40. The clamping device 40 may also be carried by an endoscope or it may be tugged into the stomach by a string that goes around a pulley anchored in the stomach as will be explained later. Preferably it is pushed along by an attached pusher element 90 which may include actuating or connecting elements as shown in FIG. 7b. The position and angle of attachment of the pusher element 90 relative to the clamping device 40 may be important in order to properly position the clamp in the stomach. This angle may be adjustable or may be fixed.

When the clamping device 40 enters the stomach, preferably an endoscope is then inserted at least to the lower esophageal sphincter 18 to help position the clamp. Positioning may be accomplished by torquing the pusher element 90 and/or manipulating the connecting elements 82 of the grasping elements 78. As shown in FIG. 8, the desired location for the clamping device 40 is to have one end 92 at the fundus 20 and the other end 94 near the lesser curvature. If four grasping elements 78 are used as shown, the clamp may be easier to align. With just two grasping elements 78, there may be a tendency for the clamping device 40 to tilt 90° to align with the plane intersecting the points of attachment of the grasping elements 78 to the anterior and posterior walls of the stomach 10.

In any case, once the clamping device 40 is positioned, it may be held in place by applying traction to the pusher element 90. The idea is to use the anatomy of the stomach 10, in particular, the angle of His 96 and the cardia 22, to wedge one end 92 of the clamping member 40. This end 92 of the clamping member 40 can then be used as a pivot point. With one end 92 essentially fixated at the cardia 22 and angle of His 96, pulling on the pusher element 90 will keep the clamping member 40 in correct alignment and position. As shown in FIG. 9, once the clamp is in place, and possibly being held by traction on the pusher element 90 in the direction of arrow A, the grasping elements 78 can be pulled, either individually or in groups, in order to drag a flap of tissue from an anterior stomach wall 100 and a posterior stomach wall 102 through the clamping member 40. Once tugging is complete, which may be confirmed visually with an endoscope, the grasping elements 78 are released and the pusher element 90 is detached from the clamping member 40.

Over the next few days or weeks, the clamping member 40 may induce ischemia in the tissue flaps and induce muscle-to-muscle and serosa-to-serosa healing of the anterior 100 and posterior 102 walls. This technique for clamp-induced securement of tissue to tissue was disclosed in a co pending U.S. Non Provisional application Ser. No. 11/418,691, filed May 6, 2006 entitled “Methods and Apparatus for Creating a Wall-to-Wall Adhesion from within an Organ Having a Lumen” the entire contents of which are included by reference.

One important consideration is the placement of the grasping elements 78. In the example shown in FIG. 8, the graspers were shown further down the stomach, below the clamping/partition site. This was to clearly show the basic method. In reality, it would be preferable to grasp closer to the site of the desired partition. Otherwise, if grasped farther down the stomach as shown in FIG. 8, after the grasping elements 78 are pulled through the clamping member 40, and the clamping member 40 and grasping elements 78 are released, the clamping member may tend to be dislocated to where stretching of the stomach is minimized. This location may be back to the point where the peak of the anterior 100 and posterior 102 flaps were pulled from. So, the preferred location for grasping member 40 in the case of an anti-obesity partition would be more like at point A as shown in FIG. 10 when two grasping elements 78 are utilized or a points B when a four grasping elements 78 are utilized.

It will be appreciated that the beam elements 42a and 42b could be made up of magnets, or could use magnets to create or augment the clamping force. The clamping force of the clamping member 40 could be pre-loaded by a retainer such as element 48 in FIG. 2 or some other force-generating means, and member 40 could be held open by use of a spacer element or multiple spacers, which are then pulled-out after the tissue is pulled through the clamp, thereby activating the clamping force against the tissue.

The ideal clamp design has rounded edges so it is easy to pass through the esophagus without injury, and so when it is in traction it does not injure the fundus or cardia or lesser curvature. Preferably the clamp is comprised of at least some elements that are biodegradable, so that after a period of time, the parts will lose their strength, fall apart, and the remnants will pass through the GI tract without incident. Some parts may not be biodegradable, such as hinge pins and the like which require higher strength than biodegradable materials may provide. Such non-degradable parts will have smooth features and be small enough to pass without incident.

Ideally, the clamping member 40 should have features which allow the tissue to be pulled easily through the clamping member 40, such as a chamfered leading edge 108 to reduce tissue trauma. It is also preferable that clamping member 40 resists backward movement of the tissue once it is pulled through the clamping member. Many solutions to this are readily known to those skilled in the art. Examples are shown in FIGS. 11A-C whereby clamping member 40 utilizes a retention element 110 of various designs. In FIG. 11A the retention element 110 is a tooth 112 that, as tissue is pulled in the direction of the arrows by the grasping elements 78 through the clamping member 40, the tooth permits the tissue to freely pass. However once tension on the tissue is released as the grasping elements 78 are released, the tooth digs into the tissue and retards backwards movement. The device may utilize multiple teeth or rows of teeth as necessary. The teeth may also be longer elements as shown in FIG. 11B that utilize long teeth 114a and 114b and pivot points 116 and 117 that actively pivot and pierce the tissue. In this embodiment, the tissue may get pierced and pinned against the opposite long tooth if the tissue moves backwards as shown in FIG. 11C.

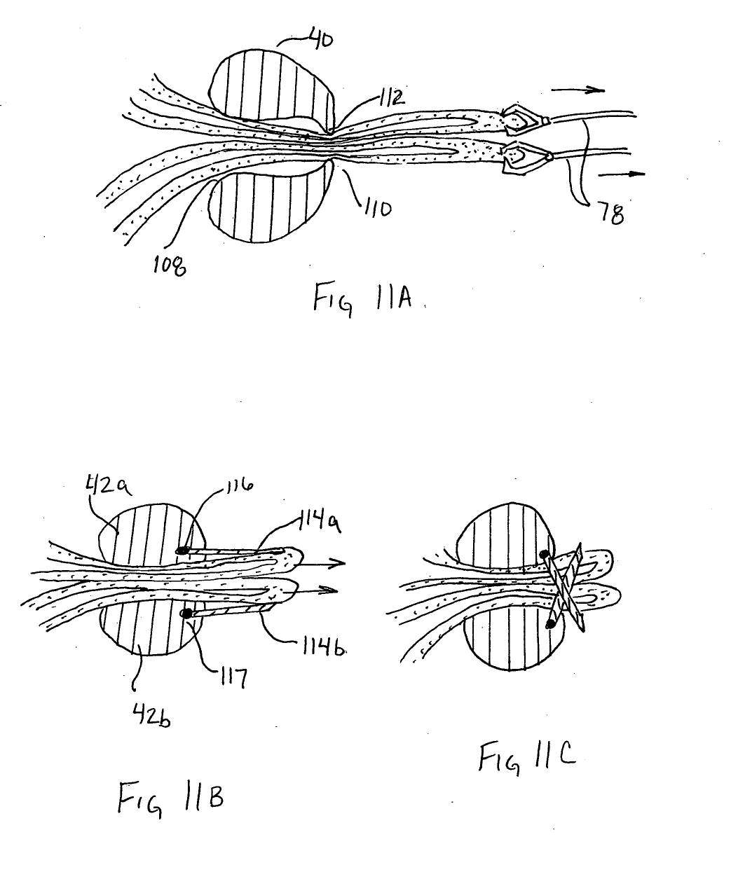

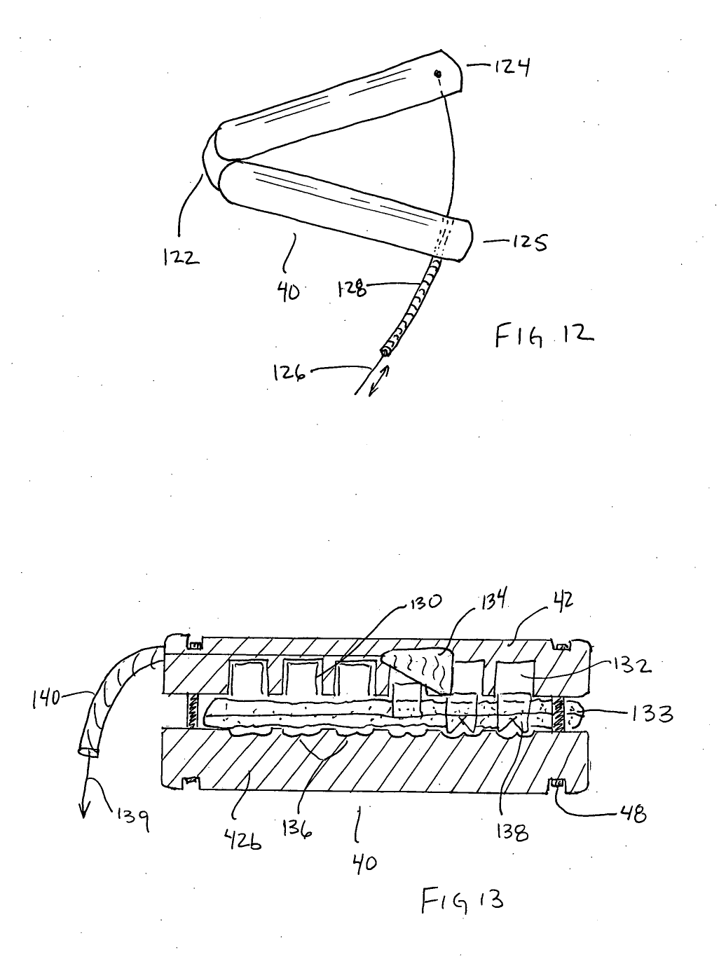

The clamp may have a user-activated clamping mechanism which may augment or replace the elastomeric or spring member discussed previously. For example as shown in FIG. 12, one end 120 of the clamping member 40 has a hinge 122. This hinge 122 could be a hinge and hinge pin design, or a living hinge as discussed previously, or it may utilize an elastomeric or spring element. The opposite ends 124 and 125 are essentially open and utilize a user-activated pull-wire to close. The pull-wire mechanism may be similar to that used in biopsy forceps; i.e., a central pull-wire 126 housed in a coil-spring 128, wherein coil spring 128 provides column strength to push against the pulling force of the pull-wire 126. It will be apparent that more substantial clamping force may be possible with the pull-wire approach as opposed to a passive non-user activated design. It will be further apparent that two pull-wires could also be used, with one on each end of the clamping member 40. The two pull-wire embodiment may provide for a more evenly distributed force across the clamping surface. The pull-wire 126 and coil 128 may need to have a release mechanism so they could be detached after clamping. This could be done with a screw-in anchor that is unseated by twisting the pull-wire 126 and coil spring 128. Or there could be a separate release wire, which when pulled, would unseat the pull-wire 126, allowing it to be removed, along with the coil spring 128. Alternatively, the pull-wire 126 could have a certain threshold of pull force above which the pull-wire 126 pops out of its anchor. That would provide an upper-end to the applied clamping force which might be beneficial. In embodiments where pull-wire 126 and coil-spring 128 are removable, clamping member 40 would incorporate a catch mechanism such as demonstrated by elements 66 and 68 shown in FIG. 3, or equivalent, in order to hold one or both ends of the clamping member 40 closed after the pull-wire 126 has been actuated and removed.

The clamping member 40 may have additional elements to further improve the healing response. These elements may be spikes, needles, wires, blades, teeth, pins or the like, and may be applied to the tissue passively, as in the case of the teeth shown in FIGS. 11A-C, or may be actively driven into the tissue. One simple way of driving elements in the tissue is by pulling a wedge across one or more tissue-piercing elements loaded inside either or both of the elongated beams as shown in FIG. 13. In this Figure, a clamping member 40 is shown with an integrated stapler system. In this example, rows of staples are incorporated into one side of the clamp, and forming anvils are located on the opposite side. The clamping member 40 is shown with two elongated beams 42a and 42b as previously described with unformed staples 130 recessed into pockets 132 formed in the upper beam 42a. The unformed staples 130 are slidably loaded into these pockets 132 and a wedge 134 is dragged across the staples 130 to force them out through the tissue 133 with the ends of the staples formed by the anvils 136 positioned opposite in the lower beam member 42b. The wedge may be activated by a pull-wire 139 encased in a spring coil 140. The formed staples 138 are formed across the folds of the organ wall and secure the two folds together. Multiple staples can be seen in FIG. 13 for it has been found that multiple staples secure the folds better than a single staple. Staplers such as this require significant force, so it is preferred that a fixed/ratcheting clamping member 40 be used as shown in FIG. 3 instead of or along with the elastomeric retainer elements 48 shown. In addition to staples it will be appreciated that wires, pins, needles, teeth, tags, tie wraps etc. may be driven into the tissue using similar or alternative mechanisms.

In the configuration disclosed thus far, the flaps of tissue that are pulled through the clamp that extend beyond the clamping surface of clamping member 40 will necrose, erode and eventually pass. In the case where the application is used to create a gastric restriction, the flaps, prior to necrosing, may initially provide an advantage in creating additional restriction to food flow and a feeling of fullness. Therefore, an alternative preferred embodiment comprises a clamp device which has an interrupted clamping surface as shown in FIG. 14. The intent is to allow enough blood supply to the flaps so that they remain viable and intact and therefore continue to provide resistance to food flow. One design factor to consider is the width of the interrupted clamping sections, indicated as “w” in the FIG. 14. After the clamp has eroded through the flaps and the clamp material has been biodegraded, there will be “windows” through the flap/partition proportioned to the width of the clamping surfaces. These windows should be small enough to minimize the amount of food that flows through them. By way of example, the windows may be about 2 mm to about 12 mm. It will be appreciated that these window features may be used advantageously to adjust the amount of food flow allowed. For example, by making the windows small enough, only liquid will flow through them, whereas by making them large, a portion of the food stream may flow through them. It will be appreciated that some or all of the windows may be plugged or unplugged with removable silicone plugs delivered endoscopically in order to decrease or increase food flow, respectively.

The flaps may also in certain cases provide too much resistance to the flow of food, so it may be desirable to cut them off. This may be done using auxiliary tools after deployment of the clamp, using tools such as a cauterizing sphincterotomy wire or knife, or it may be done by the clamp itself by incorporating a cutting element in the clamp. In one such embodiment, the cutting element may extend across one or more of the proximal edges of the clamping surfaces, similar to the embodiment shown in FIG. 11A, only instead of element 112 comprising one or more teeth, it comprises a sharp edge or pair of sharp edges. Alternatively, the cutting element may be a sliding blade 150 that is moved across the long axis of the clamping member 40, using a pull-wire 152 very similar to that shown in FIG. 13. In this case the wedge 134 has been replaced with the sliding blade 150, whose blade extends out through the clamped tissue. It will be appreciated that the cutting of the tissue may stimulate a more aggressive healing response, which may improve the degree of muscle-to-muscle healing that occurs between the clamped tissue layers. The sliding blade 150 may be centered along the longitudinal axis of the beam or it may be off axis. In yet another embodiment, a cutting element may be configured to pull through the center of the two opposed flaps of tissue, with a blade on the top and bottom of the element arranged to cut through both flaps. In a variant of this embodiment, the blades may be of insufficient depth to cut through the flaps, and therefore configured only to create an injury down to a certain depth of tissue (e.g., through the mucosa to the muscle layer) in order to provoke an aggressive muscle-to-muscle healing response without cutting off the flaps. It will be appreciated that alternatives to a blade-type cutting element may be readily conceived to create a similar injury, such as a ball with spikes on it, a rasp element, or the like. The latter embodiments would not require axial alignment as they are drawn through the flaps of tissues, and may therefore be simpler to employ.

It will be appreciated that multiple clamping surfaces and/or devices may be used to create a wall-to-wall adhesion. For example, two clamps may be used or the clamps may be different sizes or shapes with different purposes in that they may have different clamping properties and/or one may have a cutting element, or one may have both. The use of more than one clamping member 40 approach may improve the ability to keep the tissue from sliding back through the clamp or clamps.

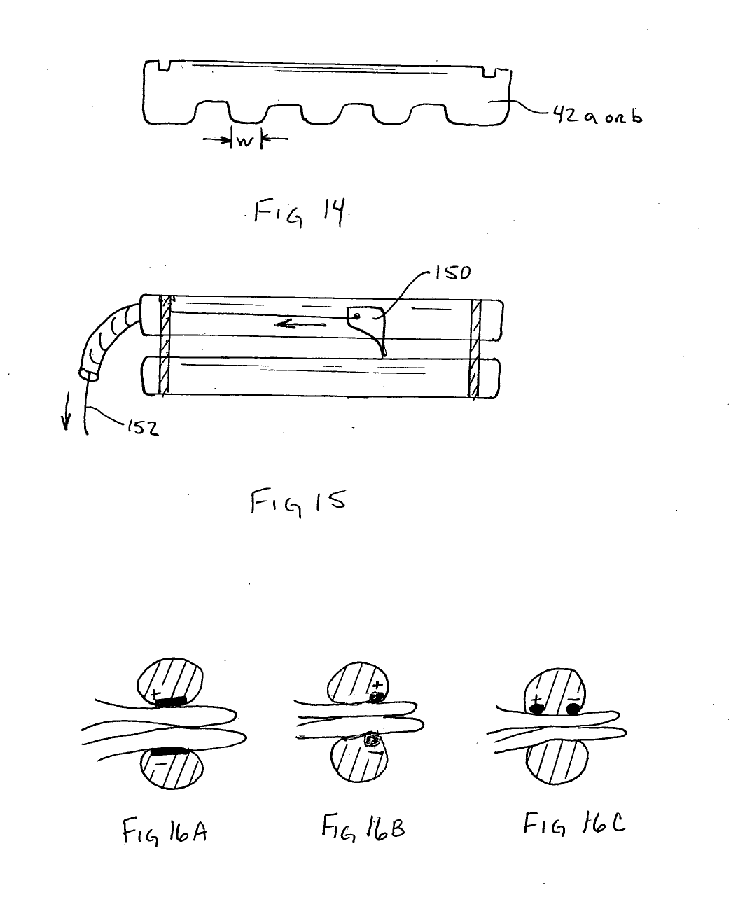

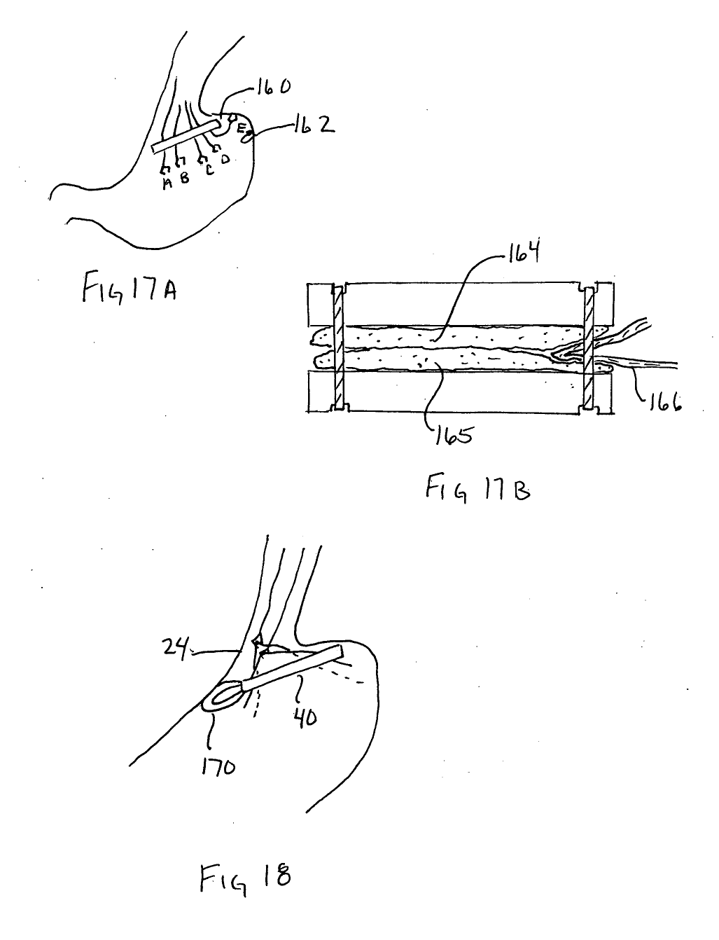

To enhance the adhesion of the clamped tissue layers, it may be beneficial to use one or more of the following to induce thermal injury to the folded tissue. Thermal heating using a heated filament, resistive heating induced by passing electrical current such as Radio Frequency (RF) through the tissue or microwave heating may be utilized and applied as a separate step or included in the clamping member design. Many configurations of electrode shape and location are apparent to those skilled in the art. For example FIG. 16A-C shows several configurations of electrodes that might be suitable for use in combination with the clamping member 40. It will be appreciated that the design in FIG. 16B has the benefit of inducing a heated/cauterized tissue zone on the flap side, leaving more tissue between the clamped surfaces for potential healing. The current and/or voltage applied to the tissue may be either DC or AC, as is well understood by those skilled in the art. Further, the energy delivery electrodes may be paired as shown in FIGS. 16A and 16C. These type of electrodes are sometimes referred to as “bipolar”, or there may be single electrode or an array of electrically-common electrodes located on the clamping member 40 and a remote electrode attached to the patient's body at a separate location. The latter configuration is often called “monopolar”. The energy delivery electrodes may also be the same as the cutting elements described in FIG. 14, thereby enabling cutting and cauterizing with a common element. This would minimize the chance of bleeding and potentially exacerbate the inflammatory and healing response of the tissue.

With the methods and devices described for creating a partition in the upper stomach, there is a chance that a significant (≧10 mm diameter) residual opening 160 will remain between the end of the formed partition and the wall of the fundus 162. One method to minimize this residual opening 160 is to use the four grasper approach rather than the dual grasper approach. Alternatively, a fifth grasper could be used to grab the wall of the fundus 162 and drag it through the clamping member 40, or to drag it into a special feature on the end of the clamping member 40 designed to close off the residual opening 160. As shown in FIGS. 17A-B, the primary folds may be formed with grasping elements A, B, C and D as previously described. A fifth grasping element E may be employed to draw into the clamping member 40 a third fold of tissue to close the residual opening 160. The wall of the fundus 162 may be drawn into the clamping member 40 and secured along with the anterior fold 164 and the posterior fold 165. The fundus wall fold 166 may be clamped along with these other layers of tissue. The fundus gap 160 could also be closed with a secondary procedure whereby tissue in the gap is gathered and cinched, plicated, glued or otherwise held together. One example would be to use a device made by Boston Scientific called the Speedband Litigator, which suctions tissue into an endcap on an endoscope and then puts a rubber band around the base of the sucked-in tissue. Another choice would be the NDO Plicatior, or a smaller version thereof.

In certain cases, when an ideal partition is created near the cardia and significant flaps of tissue are clamped, too much resistance to food flow may be created resulting in inability for the patient to eat. A potential mitigation for this complication is to add a spacer element 170 to the clamping member 40 which will ensure an opening for food flow. The spacer 170 is preferably along the lesser curvature of the stomach wall 24, since it has been shown that the lesser curvature 24 is less apt to dilate over time, and therefore has been used as the primary outlet path for conventional gastric restriction procedures such as vertical banded gastroplasty (VBG). One example of such a spacer would be a silicone ring designed to remain patent. This patency could be accomplished by using a material such as silicone that has a high material durometer to keep the ring open. Other materials such as flexible wire, Nitinol wire, coiled springs and various polymeric compounds could also be used. It may be configured to pass readily in one or more pieces or to degrade on its own after the clamping member 40 has been removed or biodegraded.

It will be appreciated that in conjunction with, or in addition to, the partition-creating methods and devices disclosed herein, the method and devices disclosed and claimed on the pending patent application 2005/0055038 filed Sep. 9, 2003 entitled “Device and Method for Endoluminal Therapy” may be utilized to create a more effective restrictive outlet. Note that the same methods may be applied to an alternate or secondary outlet located along the fundus.

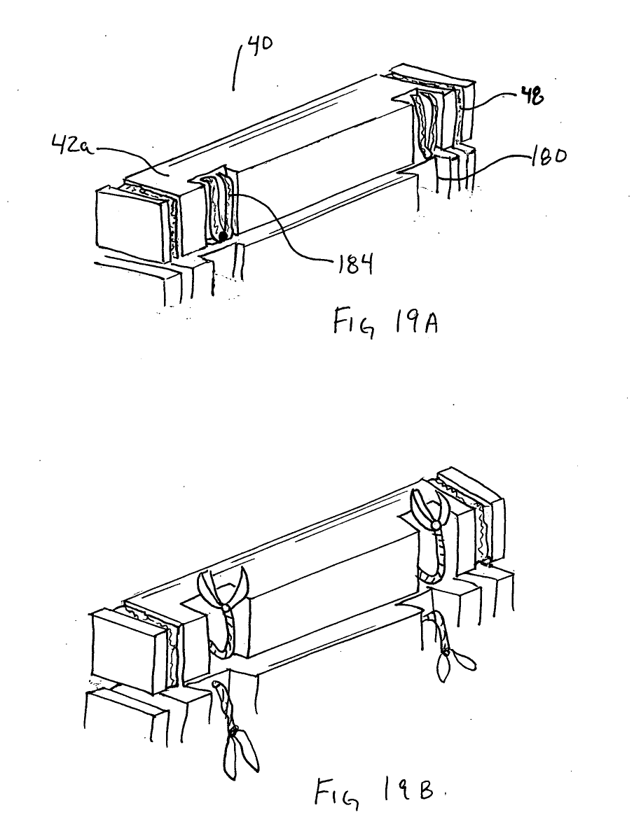

It will be appreciated that the grasping elements 78 referred to throughout this application may be integrated with the clamping member 40. Certain advantages of procedural simplification may be achieved by having grasping elements 78 loaded at spaced locations on the clamping member 40. Such grasping elements 78 are preferably in a collapsed orientation during trans-esophageal insertion, then deployed to an outwardly-directed orientation to grasp tissue. FIGS. 19A-13 illustrate an embodiment similar to that shown in FIG. 2, with the addition of grasping elements integrally located within the clamping member 40. The top beam 42a of clamping member 40 has openings 180 in the body of the beam 42a. The grasping elements 78 are shown collapsed or retracted into the openings 180 in a position suitable for introduction of the clamping member 40 into the esophageal passageway. The grasping elements 78 can be deployed from these openings 180 to grab tissue and draw it into the clamp as in previously disclosed methods. A four grasper version is shown but other numbers of grasping elements may be used.

With this method, once the clamping member 40 is positioned and the graspers are deployed, air may be removed from the stomach to collapse the walls and bring them into contact with the grasper jaws. Methods for unfolding rugal folds in order to get a deeper grasp of muscle may be utilized; such methods and devices are described and claimed in pending application 2005/0055038 previously mentioned.

Alternatively, the clamping member/grasping element assembly could be linked to a steerable component such as an endoscope, and, with the stomach at least partially insufflated, could be guided first to one wall where a pair of grasping elements 78 are deployed, and then to the other wall, where the remaining grasping elements 78 are deployed.

The adhesion of layers of clamped tissue may be improved by the insertion of an element between the layers of tissue, particularly between the opposed faces of anterior wall mucosa and posterior wall mucosa. Adhesion may also be improved by mechanically, electronically, or otherwise altering the tissues of that interface in order to trigger a more aggressive inflammatory response and subsequent healing effect. Simple mechanical removal of the mucosa and submucosa will allow better muscle-to-muscle adhesion. This may be accomplished with endoscopic mucosectomy methods and tools known to those skilled in the art. Elements that may be inserted at that tissue interface include those that have mechanical properties to disrupt the tissue, such as spike strips or strips with chemical irritants such as sodium morrhuate or silver nitrate, or the like. Elements, may also comprise mesh-type strips such as Marlex mesh (C. R. Bard) which promote tissue ingrowth, or elements made from a biomaterial such as KS (small intestinal submucosa, Cook Biotech) which has been shown to promote ingrowth in GI applications. Such elements or agents may be inserted into the interface endoscopically after the tissue flaps have been pulled through the clamping member 40, but before the clamping member is activated. Alternatively, these elements may be injected into the clamped interfaced, or wicked-in, or just applied along the exposed edge.

Throughout this disclosure, the tissue grasping elements 78 have been shown as endoscopic forceps-type devices, by way of simplicity and example only. However, it is important that the graspers bite or attack deep enough into the wall to be able to pull the tissue with adequate traction to get it through the clamp. Certain rat-tooth grasping forceps are available from companies such as Olympus and they may be adequate if used properly. Alternatives include corkscrew-type devices, such as used by the NDO Plicator device, or T-tag type anchors which are known to those skilled in the art. It will be appreciated that when manually placing the graspers and pulling on the tissue under visual guidance, multiple attempts to grasp and pull the tissue are not unreasonable since it will be obvious when the tissue has been successfully pulled into place.

Many of the embodiments thus far described utilize more than one grasping element 78. Conventionally a single accessory at a time is passed down a working channel of an endoscope. If additional accessories (such as graspers) must be used, the endoscope must be removed from the patient while leaving the grasping end 80 and the connecting means 82 in place, thereby requiring the detachment of any activation elements like handles or pull rings from the proximal end of the accessories so that the endoscope can be removed. The endoscope must be then passed back down the esophageal passageway, repositioned in the body and another accessory such as a grasper passed down the working channel and deployed. However, accessories with removable proximal elements are not typical, and the process of disassembling them and reassembling them for purposes of passing the endoscope into the patient multiple times is unwieldy. In order to simplify the process of placing multiple grasping devices sequentially, without having to remove the endoscope and without having to have a grasping element 78 with a detachable proximal activator assembly, a novel modified auxiliary working channel is hereby disclosed.

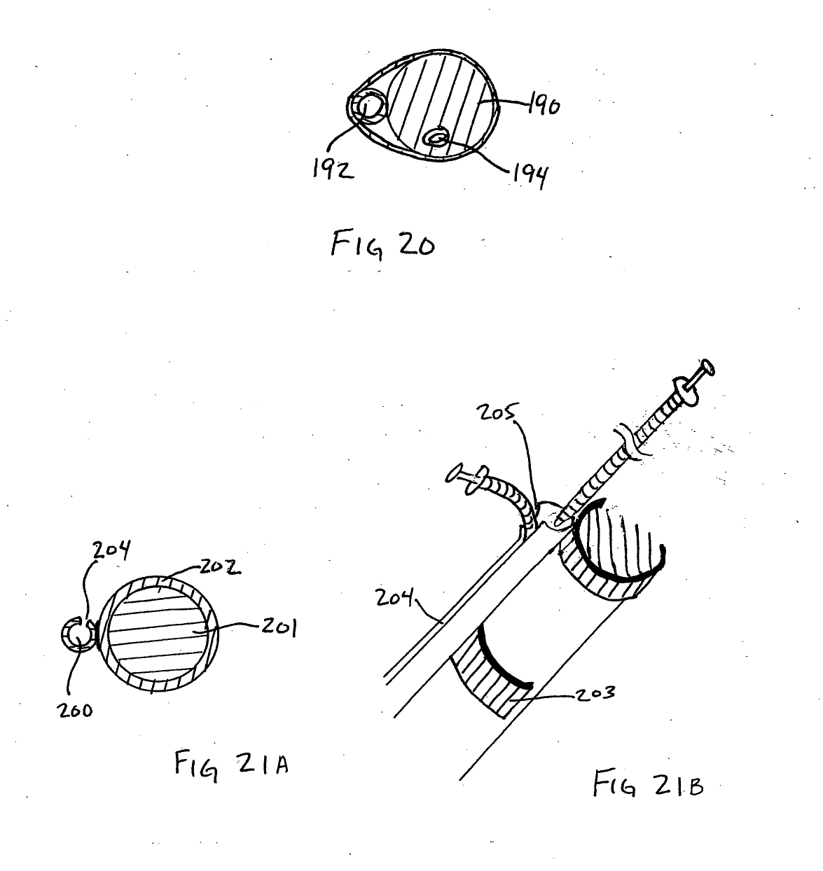

The modified auxiliary working channel is similar to that used with the Bard Endo-Cinch device, which straps onto the outside of the endoscope 190 with a cross-section shown in FIG. 20. However, this auxiliary channel 192 can only accommodate one device at a time. It will be appreciated that the conventional endoscope 190 with a single working channel 194 shown combined with the auxiliary working channel 192, could be used to place two grasping elements 78. However, the endoscope 190 would not be able to be removed to enable insertion of the clamping member 40 without having to disassemble the proximal elements of grasping elements 78. Similarly, if a two-channel scope (such as is available from Olympus) is used, that scope could not be removed either, unless the grasping elements 78 have detachable activators.

There is, therefore, a need for an auxiliary working channel which allows placement of multiple accessories sequentially and allows for the removal of the endoscope from the patient without having to remove an accessory extending through the working channel. The novel solution to this need is an auxiliary channel with the profile shown in FIGS. 21A-B. This auxiliary channel 200 is placed around a conventional endoscope 201 using a strap 202, a series of straps or a formed elastomeric clip 203. The auxiliary channel 200 has an open seam 204 that normally stays closed such that accessories are contained along the channel 200. However, when a first accessory is successfully placed and a second is desired, the proximal end of the first one can be moved out through the seam 204 at the proximal end 205 of the channel, allowing room for the tip of a second one to be inserted into the channel 200 and advanced, forcing the first accessory out through the seam 204 as it moves along. To remove an accessory from the auxiliary channel 200 in order to remove the endoscope 201, any other accessory may be used to push out the accessory already in the channel, then the other (pusher) accessory is simply pulled back out and the endoscope 201 is removed.

It is sometimes difficult to correctly identify anatomic landmarks in the stomach when performing endoscopy. For the procedure described, it is important to identify the lesser curvature correctly. To aid in this process, a catheter or guidewire with an inflatable tip may be anchored beyond the pylorus, and then held in traction in order to highlight the lesser curvature. FIG. 22 shows a stomach 10 with a catheter 210 positioned through the esophagus, through the lower esophageal sphincter 18 along the lesser curvature of the stomach 24 and through the pylorus 26. The distal end of the catheter 210 has an inflatable balloon 212 that is shown inflated. Once the balloon 212 has been inflated, the proximal end of the catheter 210 can be pulled to put the catheter 210 in traction. This catheter 210 can be seen with an endoscope and thus the lesser curvature can be identified. Such a method may be used in combination with the other novel methods described herein.

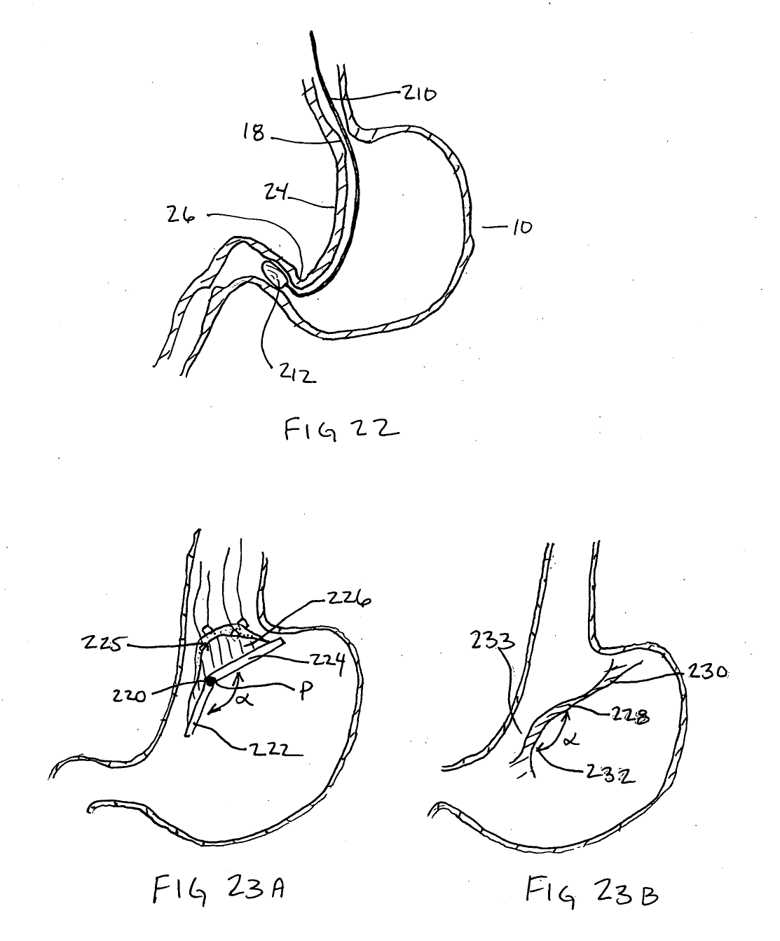

The clamping member 40 may have a more complex shape or geometry in order to form varied tissue partition configurations other than the straight-line partition as previously described. For example, the hinge feature for the clamping member 40 described previously may be used to further advantage, aside from easing trans-esophageal insertion, in order to create a bi-directional partition as shown in FIGS. 23A-B. In this application the clamping member 40 is articulated as previously described at point P by a hinge 220 at an angle α. This divides the clamping member 40 into two segments 222 and 224. The segments can be the same or different lengths depending on the partition shape desired as will be shown. Also shown are four grasping elements 78 attached to tissue flaps at four positions. Two of the grasping elements 78 are attached to a posterior tissue flap 225 and two are attached to an anterior tissue flap 226. As can be seen in FIG. 23B, the articulated clamping member 40 may create a line of tissue union that has a shape roughly corresponding to the angle of the clamping member a shown in FIG. 23A. The resultant tissue line 228 has a shape that may be more suited for a restrictive outlet. The advantage of a two-partition clamping member 40 such as this is, for example, to create one partition 230 with clamp portion 224 which essentially creates a pouch from the cardia near the lower esophageal sphincter (LES), and to create a second partition 232 with clamp portion 222 which essentially creates a restrictive outlet 233 to the pouch. Such methods are described and claimed in the pending application 2005/0055038 previously mentioned. Alternatively, the clamp may be curved to suit whatever shape partition is desired.

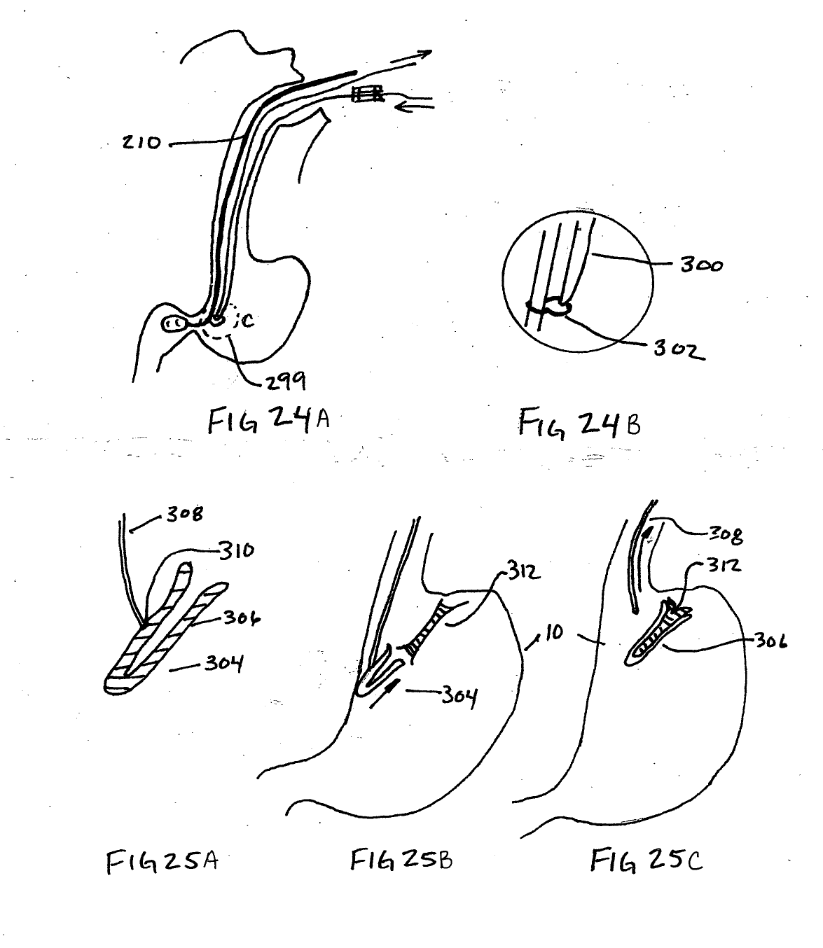

If it is desired that the partition(s) created using the methods and devices described herein be removed, conventional endoscopic tools may be used, such as RF energy-delivering tools that cut and cauterize (Gold Probe, Boston Scientific; Sphincterotome, Wilson-Cook). Alternatively, a non-RF tool such as a cutting blade or wire may be used. Another approach is to use a dilatation balloon (such as Wilson-Cook's Achalasia Dilitation balloon). The balloon would be positioned near the partition and expanded until the partition slowly rips apart. This approach may be appropriate only for certain small, thin-walled partitions under visualization. A novel method which is an additional object of the present invention is to use an endoscopically-placed clamp placed along the long axis of partition to necrose through the tissue of the partition. The important feature of this method is that the clamp is placed such that it erodes through only the “curtain” of tissue which makes up the partition and does not create an opening or ulceration in the wall of the hollow organ. This gives the method a distinct advantage over the previously described methods which use conventional tools and which run the risk of cutting or cauterizing a hole in the wall of the organ, or tearing a hole in the wall. As with the other clamp devices described elsewhere, the clamp may be made from biodegradable materials. The clamp is preferably open on one end, and has a spring element (or elastomeric element, or user-activated ratcheting element) in the non-closed end. This invention is disclosed in more detail in FIGS. 25A-C. In FIG. 25A, clamp assembly 304 is comprised of clamp 306 and guide element 308, which may be a wire or coil spring or the like. In FIG. 25B clamp assembly 304 is shown inserted through the esophagus into stomach 10, where a partition 312 had been previously formed. The opening of clamp 306 is aligned with one end of the partition and pushed onto it as shown in FIG. 25C. Once clamp 306 is positioned along the length of partition 312 and activated (if it is a user-activated clamp; otherwise it will already have a clamping force exerting onto the partition), guide element 308 may be removed from its coupling point 310 using mechanisms and methods previously described for similar elements elsewhere.

In another embodiment of the invention shown in FIGS. 24A-B, in order to pull the clamp through the esophagus, or to pull any device or accessory described, without having to have a stiff pusher element attached, a pulley-based accessory may be deployed beyond the described delivery location. In the ease of the methods and devices disclosed previously, a pulley element 299 may be added to one of the grasping elements 78, or it may be added to the balloon-tipped guidewire or catheter 210. Before either element is deployed, it may be rigged with a string 300 or other suitable device through a pulley 302 which may be a low friction structure with or without a rolling element. Once the element is anchored, the two ends of the string are outside the patient's mouth, and one end can be attached to the device to be pulled through the esophagus by pulling on the other end of the string.

It will be appreciated that while the methods and devices disclosed herein may be used in the specific manner described to create a partition in the upper stomach which is effective in treating obesity, similar methods and devices may be used to treat GERD. In particular, the creation of a partition as shown in FIG. 1 has been shown to provide an effective treatment for GERD in clinical trials (Swain CP. (1999). Endoscopic suturing. Baillieres Best Pract Res Clin Gastroenterol, 13(1):97-108). Further, the general methods and devices disclosed may be used to create partitions in any hollow organ or inside any body lumen. By way of example, this apparatus and method might be used in the colon to treat incontinence and it might be preferably useful in thinner more compliant organs. This apparatus and method might also be used for circumferential closure of an internal opening or anastomosis. It might be effective to do a sleeve resection internally or to close the stomach in gastric bypass.

Although this invention has been disclosed in the context of certain preferred embodiments and examples, it will be understood by those skilled in the art that the present invention extends beyond the specifically disclosed embodiments and/or uses of the invention and obvious modifications and equivalents thereof. Thus it is intended that the scope of the present invention herein should not be limited by the particular disclosed embodiments described above, but should be determined only by a fair reading of the claims that follow.

What is claimed is:

1. A device to secure folds of an interior wall of an organ together comprising:
at least one grasping element suitable for grasping one portion of the wall to form a first fold, at least a second grasping element suitable for grasping a second portion of the wall to form a second fold and positioning the second fold alongside the first fold with the folds in a side by side relationship, and a clamping member to secure the folds together.

2. The device of claim 1 wherein the grasping element is configured to releasably grab the wall under endoscopic guidance.

3. The grasping element of claim 2 further comprising a grasping head that is selected from the group consisting of a forcep, jaw, corkscrew, barb, hook or pincher, said grasping head is activated using a connecting member coupled to the head.

4. The clamping member of claim 1 wherein the clamping member has an open and a closed configuration, the open configuration adapted to facilitate thawing the two side by side folds into the clamping member and the closed configuration adapted to secure the folds together.

5. The clamping member of claim 4 whereby the clamping member is positioned about the grasping element such that the grasping member can move relative to the clamping member when the clamping member is in an open configuration.

6. The clamping member of claim 5 further comprising at least two parallel non-rotating elongated bars with the bars coupled to each other at both ends, the bars forming a slot through which the folds of the wall can be drawn.

7. The clamping member of claim 6 further comprising a retention element on the bars that restricts pull back of the folded walls when the clamping member is in a closed configuration.

8. The device of claim 6 wherein when the grasping element is positioned between the elongated bars, the grasping element is capable of grasping a portion of the wall and pulling the wall through slot in the elongated bars.

9. The clamping device of claim 8 wherein the elongated bars each have at least one articulated segment, the segment adapted to hinge to facilitate placement of the elongated bars through an esophageal passageway.

10. The clamping device of claim 9 wherein the bars are adapted for clamping tissue in one direction and articulating at the articulated segment in a different direction.

11. The device of claim 1 wherein the grasping element is initially housed in the clamping member for introduction to the intervention site and then is manipulated to the attachment site and activated using the connecting member.

12. A device to partition a portion of the interior space of an organ comprising:
at least one grasping element suitable for grasping one portion of a wall of the organ to initiate a first fold, at least one different grasping element suitable for grasping a second portion of a wall of the organ to initiate a second fold,
an elongate clamping member positioned about the grasping elements such that as the organ walls are drawn into the clamping member the first fold and the second fold are secured together.

13. The grasping element of claim 12 further comprising a grasping head that is selected from the group consisting of a forcep, jaw, corkscrew, barb, hook or pincher, said grasping head is activated using a connecting member coupled to the head.

14. The clamping member of claim 12 wherein the clamping member has an open and a closed configuration, the open configuration adapted so that the grasping elements can draw the organ walls into the clamping member and the closed configuration adapted to secure the folds of the wall together.

15. The elongate clamping member of claim 14 whereby the clamping member is positioned about the grasping element such that the grasping member can draw the folds of the wall into the clamping member.

16. The clamping member of claim 15 further comprising at least two parallel elongated beams with the bars coupled to each other at least at one end.

17. The clamping member of claim 16 further comprising a retention element on the beams that restricts pull back of the folded wall when the clamping member is in a closed configuration.

18. The retention member of claim 17 further comprising a sharp edge on at least one of the elongated beams.

19. The clamping member of claim 15 wherein the elongated beams each have at least one articulated segment, the segment adapted to hinge to facilitate placement of the elongated beams through an esophageal passageway.

20. The clamping device of claim 19 wherein the beams are adapted for clamping tissue in one direction and articulating at the articulated segment in a different direction.

21. A method to secure the folds of an interior wall of an organ together comprising:
grasping a first portion of the wall to form a first fold, grasping a second portion of the wall to form a second fold, positioning the second fold alongside the first fold with the folds in a side by side relationship and securing the folds together.

22. The method of claim 21 wherein grasping comprises attaching at least one grasping head to the wall in at least one discrete location and pulling on a connecting member coupled to the grasping head from outside the body to form the fold.

23. The method of claim 21 wherein securing comprises positioning at least one elongate clamping member about the side by side folds and clamping the folds together.

24. The method of claim 21 wherein securing comprises positioning at least one elongate clamping member about the connecting members,
pulling on the connecting members to draw the two side by side folds through the elongate clamping member and clamping the folds together.

25. The method of claim 24 whereby clamping comprises activating the clamping member wherein activating comprises releasing a spring, energizing a magnetic field, removing a spacer block, driving a tag, staple or pin across the folds or closing a hinge or clamp.

26. A method to create a passageway along an interior wall of the stomach comprising:
a) grasping a first portion of the wall and grasping a second portion of the wall,
b) drawing the grasped portions of wall through a clamping member such that a first and a second fold of the interior wall is formed and positioning the first fold alongside the second fold with the folds in a side by side relationship, and
c) securing the folds together with the clamping member.

27. The method of claim 26 further comprising repeating steps a, b and c to form a series of secured folds of the wall along a line running from the cardia towards the lesser curvature of the stomach.

28. The method of claim 26 wherein securing comprises clamping the folds with an elongate clamping member wherein one end of the elongate clamping member is positioned against the cardia as a pivot point such that when the folds are positioned in the clamping member, the pivot point maintains at least one end of the clamping device against the stomach wall.

29. The method of claim 28 wherein the clamping member comprises a detachable pusher element attached to the clamping member that is adapted to retain an opposite end of the elongate clamping member in a desired position while positioning the clamping member.
